(19) United States
(12) Patent Application Publication
(10) Pub. No.: US 2019/0330184 A1
(43) Pub. Date: Oct. 31, 2019

(54) 4-((6-(2,4-DIFLUOROPHENYL)-1,1-DIFLUORO-2-HYDROXY-3(5-MERCAPTO-1H-1,2,4-TRIAZOL-1-YL)PROPYL)PYRIDIN-3-YL)OXY)BENZONITRILE AND PROCESSES OF PREPARATION

(71) Applicant: Dow AgroSciences LLC, Indianapolis, IN (US)

(72) Inventors: Sarah Ryan, Indianapolis, IN (US); Qiang Yang, Zionsville, IN (US); Nicholas R. Babij, Indianapolis, IN (US); Yan Hao, Zionsville, IN (US)

(21) Appl. No.: 16/462,198

(22) PCT Filed: Nov. 17, 2017
(86) PCT No.: PCT/US17/62150

§ 371 Date: May 17, 2019

Related U.S. Application Data
(60) Provisional application No. 62/423,851, filed on Nov. 18, 2016.

(51) Int. Cl. C07D 401/06

(52) U.S. Cl. CPC C07D 401/06

(57) ABSTRACT

Provided herein is a process for the preparation of 4-((6-(2-(2,4-difluorophenyl)-1,1-difluoro-2-hydiOxy-3-(5-mercapto-1H-1,2,4-triazol-1-yl)piOpyl)pyridin-3-yl)oxy)benzonitrile.

### CROSS-REFERENCE TO RELATED APPLICATIONS

The present application claims priority under 35 U.S.C. § 119(e) to U.S. provisional patent application, U.S. Ser. No. 62/423,851, filed Nov. 18, 2016, the entire contents of which is incorporated herein by reference.

### FIELD

Provided herein is 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile and processes of preparation.

### BACKGROUND

U.S. Patent Application Ser. No. 62/163,106 describes inter alia certain metalloenzyme inhibitor compounds and their use as fungicides. The disclosure of this application is expressly incorporated by reference herein. This patent application describes various routes to generate metalloenzyme inhibiting fungicides. It may be advantageous to provide more direct and efficient methods for the preparation of metalloenzyme inhibiting fungicides and related compounds, e.g., by the use of reagents and/or chemical intermediates which provide improved time and cost efficiency.

### SUMMARY OF THE DISCLOSURE

Provided herein is the compound 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I) and processes for its preparation. In one embodiment, provided herein, is a process for the preparation of the compound of the Formula I:

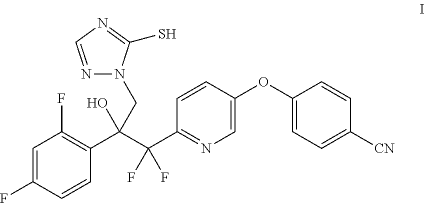 [SMILES: N#Cc1ccc(Oc2ccc(C(F)(F)C(O)(Cn3ncnc3S)c3ccc(F)cc3F)nc2)cc1]

which comprises contacting a compound of Formula II with elemental sulfur.

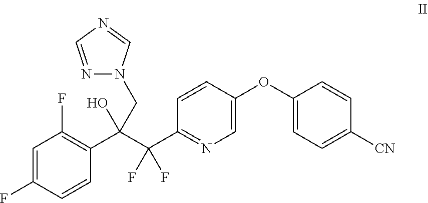 [SMILES: N#Cc1ccc(Oc2ccc(C(F)(F)C(O)(Cn3cncn3)c3ccc(F)cc3F)nc2)cc1]

The term “halogen” or “halo” refers to one or more halogen atoms, defined as F, Cl, Br, and I.

The term “organometallic” refers to an organic compound containing a metal, especially a compound in which a metal atom is bonded directly to a carbon atom.

Room temperature (RT) is defined herein as about 20° C. to about 25° C.

Throughout the disclosure, references to the compounds of Formula I-II are read as also including optical isomers and salts. Specifically, when compounds of Formula I-II contain a chiral carbon, it is understood that such compounds include optical isomers and racemates thereof. Exemplary salts may include: hydrochloride, hydrobromide, hydroiodide, and the like.

Certain compounds disclosed in this document can exist as one or more isomers. It will be appreciated by those skilled in the art that one isomer may be more active than the others. The structures disclosed in the present disclosure are drawn in only one geometric form for clarity, but are intended to represent all geometric and tautomeric forms of the molecule. For example, the chemical structures of Formulas I and Ia are tautomeric forms of the same molecule.

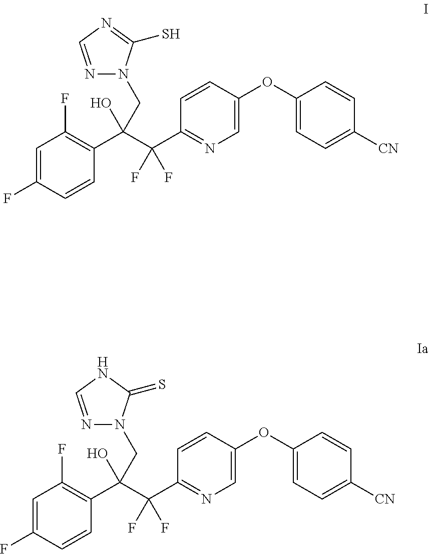 [SMILES: N#Cc1ccc(Oc2ccc(C(F)(F)C(O)(Cn3ncnc3S)c3ccc(F)cc3F)nc2)cc1 ; N#Cc1ccc(Oc2ccc(C(F)(F)C(O)(Cn3nc[nH]c3=S)c3ccc(F)cc3F)nc2)cc1]

The embodiments described above are intended merely to be exemplary, and those skilled in the art will recognize, or will be able to ascertain using no more than routine experimentation, numerous equivalents of specific processes, materials and procedures. All such equivalents are considered to be within the scope of the invention and are encompassed by the appended claims.

### DETAILED DESCRIPTION

### 4-((6-(2-(2,4-Difluorophenyl)-1,1-difluoro-2-hydroxy-3-(5-mercapto-1H-1,2,4-triazol-1-yl)propyl)pyridin-3-yl)oxy)benzonitrile (I) is Provided Herein and May be Prepared from 4-((6-(2-(2,4-difluorophenyl)-1,1-difluoro-2-hydroxy-3-(1H-1,2,4-triazol-1-yl)propyl)pyridin-3-yl)oxy)benzonitrile (II) as Shown in Example 1

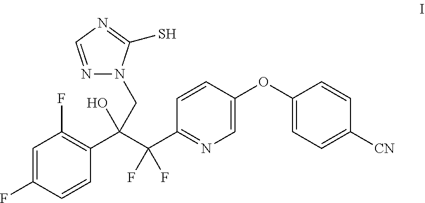 [SMILES: N#Cc1ccc(Oc2ccc(C(F)(F)C(O)(Cn3ncnc3S)c3ccc(F)cc3F)nc2)cc1]

### Example 1: Preparation of 4-((6-(2-(2,4-difluorophenyl)-1,1-difluoro-2-hydroxy-3-(5-mercapto-1H-1,2,4-triazol-1-yl)propyl)pyridin-3-yl)oxy)benzonitrile (I)

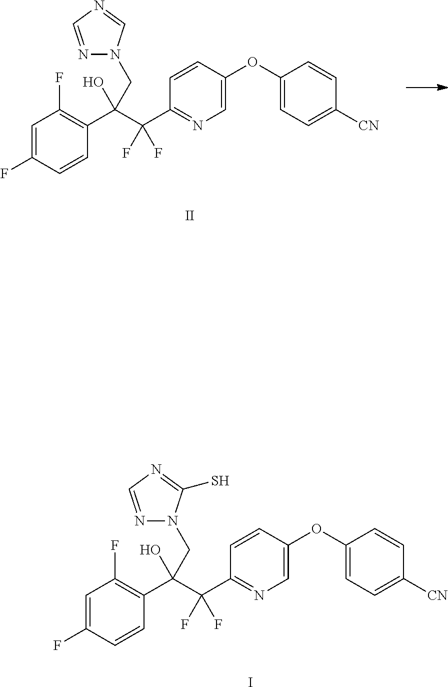

### Method A: Bubbling Air Through the Reaction Mixture.

4-((6-(2-(2,4-Difluorophenyl)-1,1-difluoro-2-hydroxy-3-(1H-1,2,4-triazol-1-yl)propyl)pyridin-3-yl)oxy)benzonitrile (II) (2 g, 4.26 mmol) and elemental sulfur (1.4 g, 43 mmol) were suspended in NMP (9 mL) in a 3-neck 100-mL round bottom flask. The flask was fitted with a thermocouple and an air condenser. A needle was inserted from a compressed air inlet line and air was gently bubbled through the reaction mixture during the course of the reaction. The reaction mixture was stirred at 180° C. for 4 h. After this time the reaction mixture was cooled to 160° C. and maintained at this temperature with stirring for an additional 12 h. After this time the mixture was analyzed by HPLC, which indicated complete consumption of starting material. The reaction mixture was cooled to room temperature and the air purge was turned off. The mixture was diluted with EtOAc (100 mL) and filtered through a Celite® pad. The EtOAc layer was washed with brine (20 mL) and water (20 mL). The organic layer was dried over anhydrous Na₂SO₄, filtered and the filtrate was concentrated to a black oil that was purified by silica gel column chromatography (80 g silica) eluting with 30-60% EtOAc/hexanes. The pure fractions were concentrated to provide 1.52 g of the desired product (I) (71% yield). ¹H NMR (400 MHz, CDCl₃) δ 11.49 (s, 1H), 8.47 (d, J=2.6 Hz, 1H), 7.74-7.61 (m, 3H), 7.56 (d, J=8.7 Hz, 1H), 7.50-7.34 (m, 2H), 7.13-6.97 (m, 2H), 6.88-6.57 (m, 2H), 5.94 (s, 1H), 5.25 (d, J=2.6 Hz, 2H).

### Method B: Bubbling Nitrogen Through the Reaction Mixture.

To a 3-neck 2000 mL flask equipped with a mechanical stirrer, a nitrogen inlet, and a temperature probe was charged with 4-((6-(2-(2,4-difluorophenyl)-1,1-difluoro-2-hydroxy-3-(1H-1,2,4-triazol-1-yl)propyl)pyridin-3-yl)oxy)benzonitrile (II) (60 g, 121 mmol) and N-methyl-2-pyrrolidinone (NMP, 350 mL). Sulfur (38.9 g, 1214 mmol) was added. A glass tube was inserted from the nitrogen inlet line and nitrogen was gently bubbled through the reaction mixture during the course of the reaction. The reaction mixture was heated at 180° C. for 5 h, after which it was cooled down to 20° C. and the reaction mixture was diluted with MTBE (700 mL). Activated carbon (90 g, Darco KB; about 100 mesh) was added and the suspension was stirred for 1 h. The mixture was filtered through a pad of Celite®, and the filter cake was rinsed with MTBE (2×800 mL). The combined filtrate and rinses were washed with brine (3×1000 mL) and the first aqueous wash was extracted with MTBE (400 mL). The combined organic layers were extracted with 1 N NaOH (2.4 L) and the aqueous layer was re-extracted with DCM (2×1.2 L). EtOAc (2.4 L) was added to the aqueous layer and the pH was adjusted to 3 with HCl (37 wt %, 200 mL). The organic layer was separated and washed with saturated NaHCO₃ (2×1200 mL) and brine (3×1000 mL), dried over anhydrous Na₂SO₄, and filtered. The filtrates were concentrated to provide an off-white foam (47 g). The material was suspended in 50% EtOAc/hexanes (1440 mL) for 15 min at 55° C. and filtered. The solid was further slurried in 500 mL of EtOAc/hexanes (1:1) and filtered. The filter cake was further dried under vacuum to afford an off-white solid (25 g). The filtrates were combined and concentrated to give 25 g of an off-white solid. 720 mL of EtOAc/hexanes (1:1) was added and the suspension was stirred at 50° C. for 30 min. The suspension was cooled to 20° C., stirred for 1 h, and filtered to give a second crop of the desired product as an off-white solid (15 g). The combined yield was 62%. mp: 205-208° C. ¹H NMR (400 MHz, DMSO-d₆) δ 13.59 (s, 1H), 8.46 (d, J=2.7 Hz, 1H), 8.18 (s, 1H), 7.91 (d, J=8.3 Hz, 2H), 7.71 (dd, J=8.7, 2.7 Hz, 1H), 7.63 (d, J=8.7 Hz, 1H), 7.37 (q, J=8.3 Hz, 1H), 7.18 (dd, J=28.1, 9.5 Hz, 3H), 6.95 (t, J=7.4 Hz, 1H), 6.42 (s, 1H), 5.20-4.92 (m, 2H). ¹³C NMR (126 MHz, DMSO) δ 166.52, 161.52 (dd, J=321.3, 12.6 Hz), 159.55 (dd, J=326.3, 12.6 Hz), 159.86, 152.49, 147.24 (t, J=27.7 Hz), 140.89, 139.30, 134.90, 131.82 (dd, J=9.9, 5.0 Hz), 127.56, 124.42 (t, J=4.2 Hz), 120.37 (dd, J=12.2, 3.6 Hz), 119.14 (t, J=254.5 Hz), 118.81, 118.50, 110.66 (dd, J=20.8, 3.1 Hz), 106.45, 104.05 (dd, J=29.0, 25.6 Hz), 78.48 (dt, J=153.7, 3.8 Hz), 49.76 (d, J=8.8 Hz).

Method C: Bubbling Nitrogen Through the Reaction Mixture with Alternative Isolation.

4-((6-(2-(2,4-Difluorophenyl)-1,1-difluoro-2-hydroxy-3-(1H-1,2,4-triazol-1-yl)propyl)pyridin-3-yl)oxy)benzonitrile (II) (5 g, 10.65 mmol) and elemental sulfur (3.42 g, 107 mmol) were suspended in NMP (40 mL) in a 3-neck 250-mL round bottom flask. The flask was fitted with a thermocouple and an air condenser. A needle was inserted from a compressed nitrogen inlet line and nitrogen was gently bubbled through the reaction mixture during the course of the reaction. The reaction mixture was stirred at 180° C. for 7 h. MTBE (100 mL) was added to the reaction mixture and it was filtered through a pad of Celite®. To the filtrate was added water (100 mL) and the mixture was filtered again through Celite® and the phase separated. The aqueous layer was extracted with MTBE (50 mL) and the combined organic layers were extracted with 1 M NaOH (100 mL). The aqueous layer was washed with DCM (50 mL) then extracted with ethyl acetate (100 mL). The ethyl acetate layer was washed with brine (50 mL). To the organic layer was added water (50 mL) and the mixture was acidified to pH 5 using 2 M HCl. The phases were separated and the organic layer was dried over sodium sulfate, filtered, and concentrated to half of its volume. The product was crystallized from the ethyl acetate solution by addition of heptane (50 mL) giving the desired product as a white solid (3.77 g, 69% yield). Spectral data matched those described above.

Suitable solvents for use in this process step may be selected from at least one of dimethylsulfoxide (DMSO), N,N-dimethylformamide (DMF), N,N-dimethylacetamide (DMAc), sulfolane, and N-methyl-2-pyrrolidone (NMP).

This process step may be conducted at temperatures from about 50° C. to about 200° C., or from about 120° C. to about 180° C.

What is claimed is:

1. A method of making a compound of Formula I 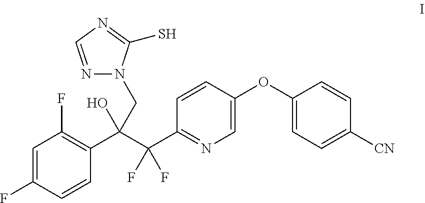 [SMILES: N#Cc1ccc(Oc2ccc(C(F)(F)C(O)(Cn3ncnc3S)c3ccc(F)cc3F)nc2)cc1]
comprising the step of contacting a compound of Formula II 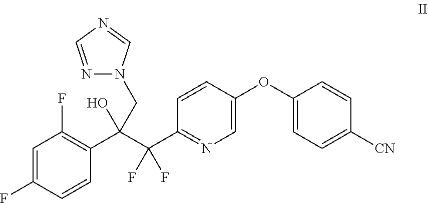 [SMILES: N#Cc1ccc(Oc2ccc(C(F)(F)C(O)(Cn3cncn3)c3ccc(F)cc3F)nc2)cc1]
with elemental sulfur to form a mixture.

2. The method of claim 1, further comprising passing a gas through the mixture during the contacting.

3. The method of claim 2, wherein the gas is selected from air and nitrogen.

4. The method of claim 1 further comprising a solvent selected from at least one of dimethylsulfoxide, N,N-dimethylformamide, N,N-dimethylacetamide, sulfolane, and N-methyl-2-pyrrolidone.

5. The method of claim 1 wherein the contacting is carried out between about 50° C. and about 200° C.

6. The method of claim 1 wherein the contacting is carried out between about 120° C. and about 180° C.
